(19) United States
(12) Patent Application Publication
(10) Pub. No.: US 2022/0265560 A1
(43) Pub. Date: Aug. 25, 2022

(54) CAPSULE DEVICE HAVING IMPROVED SELF-RIGHTING ABILITY

(71) Applicant: Novo Nordisk A/S, Bagsvaerd (DK)

(72) Inventors: Mikkel Wennemoes Hvitfeld Ley, Broenshoej (DK); Morten Revsgaard Frederiksen, Copenhagen K (DK); Brian Jensen, Broenshoej (DK)

(21) Appl. No.: 17/627,226

(22) PCT Filed: Jul. 22, 2020
(86) PCT No.: PCT/EP2020/070736

§ 371 Date: Jan. 14, 2022

(30) Foreign Application Priority Data
Jul. 22, 2019 (EP) 19187663.0

(51) Int. Cl. A61K 9/48; A61M 31/00; A61B 5/00; A61M 5/20

(52) U.S. Cl. CPC A61K 9/4808; A61M 31/002; A61B 5/6861; A61M 5/2033

(57) ABSTRACT

A capsule device suitable for insertion into a lumen of a patient, the lumen having a lumen wall, wherein the capsule device (100, 200, 300, 400, 500) comprises a) a capsule housing (110, 120, 210, 220) having an outside shape formed as a rounded object and defining an exterior surface, and b) a tissue interfacing component (130, 230) disposed relative to the capsule housing (110, 120, 210, 220), the tissue interfacing component (130, 230) configured to interact with the lumen wall at a target location, wherein the capsule device is configured as a self-righting capsule having a geometric center and a center of mass offset from the geometric center along a first axis, wherein when the capsule device (100, 200, 300, 400, 500) is supported by the tissue of the lumen wall while being oriented so that the centre of mass is offset laterally from the geometric center the capsule device experiences an externally applied torque due to gravity acting to orient the capsule device with the first axis oriented along the direction of gravity to enable the tissue interfacing component (130, 230) to interact with the lumen wall at the target location, wherein at least a portion of the exterior surface of the capsule device (100, 200, 300, 400, 500) has a surface property exhibiting one or more surface properties selected from the group consisting of a surface coating, a surface roughness, a surface geometry, and a surface micro-geometry, and wherein said surface property is selected to provide low friction, such as low static friction, ensuring slipping movement of the capsule device relative to the tissue of the lumen wall when said externally applied torque due to gravity acts on the capsule device.

### CAPSULE DEVICE HAVING IMPROVED SELF-RIGHTING ABILITY

The present invention relates to capsule devices for medical diagnosis and/or therapy, the capsule devices being adapted for insertion into a lumen of a patient, wherein the capsule devices have an inherent ability to self-orient relative to a supporting surface of a lumen wall.

### BACKGROUND OF THE INVENTION

In the disclosure of the present invention reference is mostly made to the treatment of a disease by delivery of a drug payload, such as delivery of insulin for treating diabetes. However, this is only an exemplary use of the present invention.

May people suffer from diseases, such as diabetes, which requires them to receive injections of drugs on a regular and often daily basis. To treat their disease these people are required to perform different tasks which may be considered complicated and may be experienced as uncomfortable. Furthermore, it requires them to bring injection devices, needles and drugs with them when they leave home. It would therefore be considered a significant improvement of the treatment of such diseases if treatment could be based on oral intake of tablets or capsules.

However, such solutions are very difficult to realise, since protein-based drugs will be degraded and digested rather than absorbed when ingested.

To provide a working solution for delivering insulin into the bloodstream through oral intake, the drug has to be delivered firstly into a lumen of the gastrointestinal tract and further into the wall of the gastrointestinal tract (lumen wall). This presents several challenges among which are: (1) The drug has to be protected from degradation or digestion by the acid in the stomach. (2) The drug has to be released while being in the stomach, or in the lower gastrointestinal tract, i.e. after the stomach, which limits the window of opportunity for drug release. (3) The drug has to be delivered at the lumen wall to limit the time exposed to the degrading environment of the fluids in the stomach and in the lower gastrointestinal tract. If not released at the wall, the drug may be degraded during its travel from point of release to the wall or may pass through the lower gastrointestinal tract without being absorbed, unless being protected against the decomposing fluids.

WO2018//213600 A1 discloses various self-righting articles, such as self-righting capsules, intended to be ingested by a patient into the GI tract to offer diagnosis or therapy to the patient. A self-righting article may be configured as a monostatic body which due to the location of the center of mass and the shape of the sell-righting article exhibits an inherent ability to self-orient in a pre-defined orientation, allowing a tissue interfacing component to locate next to a target site of a lumen wall.

The ability of a self-righting capsule to self-orient relative to a supporting surface, and the ability to remain positioned in the intended orientation once it has been obtained, is dependent on various factors. During design of the self-righting capsule, in order to accommodate different diagnostic and/or therapeutic actions of the self-righting article, various design factors must be taken into consideration. The capsule must provide an adequate self-righting ability while still enabling the intended diagnostic and/or therapeutic action or function. Also, the overall size of the capsule may in many applications be critical. For example, when aiming for sufficient loading capacity of a capsule having a small size, it may be challenging to obtain a desired centre of mass for the article, and this may result in a reduced ability to self-orient.

Having regard to the above, it is an object of the present invention to provide a self-righting capsule device for insertion into a lumen of a patient, wherein in a situation of use, when the capsule device is supported by tissue at a target location in the lumen, the capsule device exhibits an improved ability to enter into a pre-defined orientation, and to maintain the predefined orientation once the pre-defined orientation has been assumed. A further object of the present invention is to provide increased freedom in designing a self-orienting capsule while obtaining a superior ability to self-orient.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in a first aspect of the invention a capsule device is provided which is suitable for insertion into a lumen of a patient, the lumen having a lumen wall, wherein the capsule device comprises:
a capsule housing having an outside shape formed as a rounded object and defining an exterior surface, and
a tissue interfacing component disposed relative to the capsule housing, the tissue interfacing component configured to interact with the lumen wall at a target location, wherein the capsule device is configured as a self-righting capsule having a geometric center and a center of mass offset from the geometric center along a first axis, wherein when the capsule device is supported by the tissue of the lumen wall while being oriented so that the centre of mass is offset laterally from the geometric center the capsule device experiences an externally applied torque due to gravity acting to orient the capsule device with the first axis oriented along the direction of gravity to enable the tissue interfacing component to interact with the lumen wall at the target location,

wherein at least one portion of the exterior surface of the capsule device has a surface property exhibiting one or more surface properties selected from the group consisting of a surface coating, a surface roughness, a surface geometry, and a surface micro-geometry, and wherein said surface property is selected to provide low friction, such as low static friction, ensuring slipping movement of the capsule device relative to the tissue of the lumen wall when said externally applied torque due to gravity acts on the capsule device.

For state of art capsules it has been suggested to configure the self-righting capsules with such density distribution, and with such geometrical shape and surface properties, that the self-righting capsule rolls without slipping relative to the mucosal tissue when a torque due to gravity acts on the self-righting capsule. Compared to state of art self-righting capsules wherein surface portions of the capsules provide a relative large coefficient of friction, the proposed property of the exterior surface according to the invention provides for slipping rotation of the capsule device relative to the supporting lumen wall. This has a marked effect on the self-righting torque exerted by gravity, and provides for improved self-righting of the capsule device. The improvements in self-righting may be utilized to ensure better and quicker self-righting, and/or may be utilized for providing improved design-freedom for the capsule device, such as the freedom in the layout and distribution of components internally in the capsule device.

Said surface property of the at least a portion of the exterior surface of the capsule device may be so selected that, when the capsule device is supported on a level surface, the low static friction ensures slipping movement of the capsule device relative to the tissue of the lumen wall when said externally applied torque due to gravity acts on the capsule device.

In some embodiments the entire capsule exterior has said surface property.

In other embodiments a lower part of the capsule device adjacent the tissue interfacing component, such as the lower half surface area of the capsule total exterior surface area, includes surface portions having said surface property.

In some embodiments the at least one portion of the exterior surface of the capsule device provides a a low-friction surface having a coefficient of static friction below 0.35, such as below 0.30, such as below 0.25, such as below 0.20, such as below 0.15, such as below 0.10, such as below 0.05, or such as below 0.02.

In some embodiments the at least one portion of the exterior surface of the capsule device provides a a low-friction surface having a coefficient of static friction between 0.01 and 0.35, preferably between 0.01 and 0.30, preferably between 0.01 and 0.25, more preferably between 0.01 and 0.20, more preferably between 0.01 and 0.15, more preferably between 0.01 and 0.10, and more preferably between 0.01 and 0.05.

In some embodiments the at least one portion of the exterior surface of the capsule device provides a a low-friction surface having a coefficient of static friction when wet between 0.01 and 0.35, preferably between 0.01 and 0.30, preferably between 0.01 and 0.25, more preferably between 0.01 and 0.20, more preferably between 0.01 and 0.15, more preferably between 0.01 and 0.10, and more preferably between 0.01 and 0.05.

In some embodiments the said surface property is selected to provide low friction for said at least a portion of the exterior surface of the capsule device with a coefficient of static friction between 0.01 and 0.35.

In other embodiments the said surface property is selected to provide low friction for said at least a portion of the exterior surface of the capsule device with a coefficient of static friction between 0.01 and 0.25.

For some embodiments, the one or more surface properties are selected as to include surface areas having a coefficient of static friction in the order of 0.01-0.20, such as 0.01-0.10, such as 0.01-0.06.

For some embodiments, the coefficient of static friction is in the order 0.02-0.05.

For some embodiments, for a polished material for the capsule device exterior surface, exemplary surface finishes may be provided in the range Ra 0.02 to Ra 0.80.

In some forms the tissue interfacing component comprises at least one of a therapeutic payload, a diagnostic device and a tissue retaining device, such as a tissue anchoring device.

In further embodiments of the capsule device the tissue interfacing component comprises or defines a therapeutic payload configured to provide release of at least a part of the therapeutic payload to the lumen wall at the target location.

The therapeutic payload may be disposed or configured disposable in the capsule device, wherein the therapeutic payload configured for being expelled from the capsule into the lumen wall at the target location.

In some forms the capsule device further comprises a delivery member disposed or disposable in the capsule device, the delivery member being shaped to penetrate tissue of the lumen wall and having a tissue penetrating end and a trailing end opposite the tissue penetrating end, wherein the delivery member is configured to deliver the therapeutic payload from a reservoir or comprises the therapeutic payload.

In some embodiments the capsule device further comprises an actuator coupled to the delivery member and having a first configuration and a second configuration, the delivery member being retained within the capsule when the actuator is in the first configuration, wherein the delivery member is configured to be advanced from the capsule and into the lumen wall by movement of the actuator from the first configuration to the second configuration.

The delivery member may in some forms be provided as a solid formed entirely from a preparation comprising the therapeutic payload, wherein the delivery member is made from a dissolvable material that dissolves when inserted into tissue of the lumen wall to deliver at least a portion of the therapeutic payload into tissue.

In other forms the delivery member is an injection needle, wherein the therapeutic payload is provided as a liquid, gel or powder being expellable through the injection needle from a reservoir within the capsule.

In some embodiments the actuator comprises an energy source associated with the delivery member, the energy source being configured for powering the delivery member for being advanced from the capsule and into the lumen wall by movement of the actuator from the first configuration to the second configuration.

The actuator may in further embodiments comprise the energy source, such a drive spring. the spring being strained or configured for being strained for powering the delivery member. The drive spring may be provided in the form as a compression spring, a tension spring, a torsion spring or a leaf spring.

In some forms the capsule device comprises a dissolvable firing member, the dissolvable firing member being at least partially dissolvable when subjected to a biological fluid, wherein the dissolvable firing member, when at least partially dissolved, permits release of energy from the energy source so that the delivery member is advanced from the capsule and into the lumen wall.

In some embodiments, the capsule device defines an ingestible capsule having a capsule housing shaped and sized to be ingested by a patient. The patient may be a human patient.

The capsule device may in different embodiments be provided configured for release of therapeutic payload from the capsule into one of a lumen wall of the stomach, a lumen wall of the large intestines and a lumen wall of the small intestines of a patient.

In a second aspect of the invention a capsule device is provided which is suitable for ingestion into a lumen of the GI tract, the lumen having a lumen wall, wherein the capsule device comprises:

a capsule housing having an outside shape formed as a rounded object and defining an exterior surface, and - a tissue interfacing component disposed relative to the capsule housing, the tissue interfacing component configured to interact with the lumen wall at a target location, wherein the capsule device is configured as a self-righting capsule having a geometric center and a center of mass offset from the geometric center along a first axis, wherein when the capsule device is supported by the tissue of the lumen wall while being oriented so that the centre of mass is offset laterally from the geometric center the capsule device experiences an externally applied torque due to gravity acting to orient the capsule device with the first axis oriented along the direction of gravity to enable the tissue interfacing component to interact with the lumen wall at the target location, wherein at least one portion of the exterior surface of the capsule device provides a a low-friction surface having a coefficient of static friction between below 0.35, such as below 0.30, such as below 0.25, such as below 0.20, such as below 0.15, such as below 0.10, such as below 0.05, or such as below 0.02.

In some embodiments the at least one portion of the exterior surface of the capsule device provides a a low-friction surface having a coefficient of static friction between 0.01 and 0.35, preferably between 0.01 and 0.25, more preferably between 0.01 and 0.20, more preferably between 0.01 and 0.15, more preferably between 0.01 and 0.10, and more preferably between 0.01 and 0.05.

In some embodiments the at least one portion of the exterior surface of the capsule device provides a a low-friction surface having a coefficient of static friction when wet between 0.01 and 0.35, preferably between 0.01 and 0.25, more preferably between 0.01 and 0.20, more preferably between 0.01 and 0.15, more preferably between 0.01 and 0.10, and more preferably between 0.01 and 0.05.

In further embodiments, any of the features mentioned in connection with the first aspect above are provided in combination with the features of the second aspect.

As used herein, the terms “drug” or “payload” is meant to encompass any drug formulation capable of being delivered into or onto the specified target site. The drug may be a single drug compound or a premixed or co-formulated multiple drug compound. Representative drugs include pharmaceuticals such as peptides (e.g. insulins, insulin containing drugs, GLP-1 containing drugs as well as derivatives thereof), proteins, and hormones, biologically derived or active agents, hormonal and gene based agents, nutritional formulas and other substances in both solid, powder or liquid form. Specifically, the drug may be an insulin or a GLP-1 containing drug, this including analogues thereof as well as combinations with one or more other drugs.

### BRIEF DESCRIPTION OF THE DRAWINGS

In the following embodiments of the invention will be described with reference to the drawings, wherein

FIG. 1 shows a cross-sectional side view of a capsule device configured for solid dose delivery, representative for both a state of art capsule device 10, and for a first embodiment 100 of a capsule device in accordance with the invention, the device assuming a pre-firing configuration,

FIGS. 2a and 2b show various geometric definitions for the capsule device shown in FIG. 1 with the device being exerted to a field of gravity and oriented for single point contact with a lumen wall in Contact point P,

FIG. 2c shows the capsule device of FIG. 1 in four different orientations,

FIG. 3a schematically illustrates the capsule device 10 of FIG. 1 in single point contact with a supporting surface, the device being configured for rolling without slipping relative to the surface,

FIG. 3b schematically illustrates the capsule device 100 of FIG. 1 in single point contact with a supporting surface, the device being configured for rolling with slipping relative to the surface,

FIG. 4a schematically illustrates the capsule device 10 of FIG. 1 in line contact with a supporting surface, the device being configured for rolling without slipping relative to the surface,

FIG. 4b schematically illustrates the capsule device 100 of FIG. 1 in line contact with a supporting surface, the device being configured for rolling with slipping relative to the surface,

FIG. 5 shows various geometric definitions and equations for the capsule device shown in FIG. 1 with the device being exerted to a field of gravity and oriented for line contact with a lumen wall in Contact point P,

FIG. 6a shows a cross-sectional side view of a second embodiment 200 of a capsule device in accordance with the invention, the device assuming a pre-firing configuration,

FIG. 6b is a contact point analysis for the second embodiment 200 in a given orientation, the figure depicting three calculations corresponding to three different levels of protrusion height (h) into a lumen wall,

FIG. 6c shows curves of calculated torque levels as a function of elevation angle (θ) for the second embodiment 200,

FIG. 7a shows a cross-sectional side view of a third embodiment 300 of a capsule device in accordance with the invention, the device assuming a pre-firing configuration,

FIG. 7b is a contact point analysis for the third embodiment 300 in a given orientation, the figure depicting three calculations corresponding to three different levels of protrusion height (h) into a lumen wall,

FIG. 7c shows curves of calculated torque levels as a function of elevation angle (θ) for the third embodiment 300,

FIG. 8 is a comparison of calculated torque levels as a function of elevation angle (θ) for the first, the second and the third embodiment respectively, the torque levels being calculated as single point contact with the lumen wall,

FIGS. 9a and 9b each shows a cross-sectional front view of a fourth embodiment of a capsule device in accordance with the invention configured for solid dose delivery, the device assuming a pre-firing configuration and a firing configuration, respectively,

FIG. 10 shows schematically three different configurations of an assembly of a ram and a solid dose delivery member for use in a capsule device according to an aspect of the invention,

FIG. 11 shows schematically four different configurations of pairs of deformable latch and retaining portion assemblies for use in firing a ram in a capsule device,

FIG. 12 shows schematically three different configurations of a capsule and ram assembly to enable solid dose delivery detachment between a solid delivery member and a ram,

FIGS. 13a, 13b and 13c each shows a cross-sectional front view of a fifth embodiment of a capsule device, the device being configured for liquid dose delivery, wherein the device assumes a pre-firing configuration, a firing configuration, and an end-of-dose configuration respectively,

FIG. 14 is a cross-sectional side view corresponding to the front view shown in FIG. 13c,

FIG. 15 shows various geometric definitions for the second embodiment capsule device 200 shown in FIGS. 6a, and

FIG. 16 is a schematic representation of a spherical capsule device indicating various geometric definitions.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The terms “assembly” and “subassembly” do not imply that the described components necessarily can be assembled to provide a unitary or functional assembly or subassembly during a given assembly procedure but is merely used to describe components grouped together as being functionally more closely related.

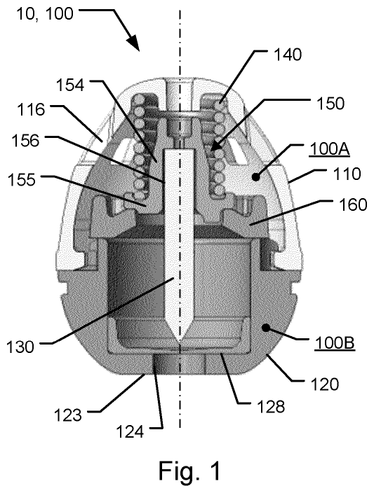

With reference to FIG. 1 a first example device representing a self-righting capsule 10 is shown.

The self-righting capsule 10 is suitable for being ingested by a patient to allow the capsule device to enter the stomach lumen, subsequently to orient relative to a wall of the lumen, and finally to deploy a solid dose drug payload for insertion at a target location in mucosal tissue of the stomach wall. The capsule device 10 utilizes some of the general principles disclosed in WO 2018/213600 A1 for enabling self-orienting of the capsule relative to the stomach wall, and to deploy a solid dose payload for drug administration.

The ingestible self-righting capsule device 10 comprises a first portion 100A having an average density, a second portion 100B having an average density different from the average density of the first portion 100A. The capsule device 10 accommodates a payload portion 130 for carrying an agent for release internally of a subject user that ingests the article. In the shown embodiment, the average density of capsule device prior to deployment is larger than that of gastrointestinal fluid, enabling the capsule device to sink to the bottom of the stomach lumen. The outer shape of the self-righting article may be a gomboc shape, i.e. a gomboc-type shape that, when placed on a surface in any orientation other than a single stable orientation of the shape, then the shape will tend to reorient to its single stable orientation.

In FIG. 1, for the shown example device 10, the capsule is shaped so that it has a central axis of symmetry. The central axis of symmetry for the device runs vertically when the bottom surface 123 of the device is facing downwards in the direction of gravity. The shown capsule device 10 includes an upper (proximal) capsule part 110 which mates and attaches to a lower (distal) capsule part 120. The upper capsule part 110 and the lower capsule part 120 together forms the capsule housing of the device. The capsule defines an interior hollow which accommodates the payload portion 130, a ram 150 forming a needle hub which holds the payload portion 130, and a firing and propulsion mechanism including an actuator configured to fire and drive forward the needle hub with the payload for drug delivery. The payload portion 130, held by payload interface portion 156 on ram 150, is oriented along a firing axis which runs coaxially with the central axis. The payload is configured for movement along the firing axis. In the shown embodiment, the upper and lower capsule parts 110, 120 form rotation symmetric parts which are substantially symmetric around the firing axis. In FIG. 1, the device 10 is oriented with the firing axis pointing vertically, and with the payload portion 130 pointing vertically downwards towards an exit hole 124 arranged centrally in the lower capsule part 120, the exit hole allowing the payload portion 130 to be transported through the exit hole and moved outside the capsule device 10. The lower part 120 includes a tissue engaging surface 123 which is formed as a substantially flat lower outer surface surrounding the exit hole 124.

In the shown example the payload portion 130 defines a solid dissolvable delivery member formed partly or entirely from a preparation comprising the therapeutic payload. In the shown embodiment, the solid delivery member is formed as a thin cylindrical rod shaped to penetrate tissue of the lumen wall, the cylindrical rod having a tissue penetrating end and trailing end opposite the tissue penetrating end. The tissue penetrating end of the rod is pointed to facilitate easy insertion into tissue of the lumen wall whereas the trailing end, in the shown embodiment, defines a truncated cylinder cut off by a 90-degree cut. A non-limiting example of a drug suitable for delivery by capsule device 400 is dried compressed API such as insulin. Due to the shape of the solid delivery member (payload portion 130) being formed as a thin rod, the delivery member is suitable for being pressed into tissue at a target location where after the delivery member starts to degrade to release drug via the mucosal tissue at the target location.

The firing and propulsion mechanism of the example device 10 includes an actuator in the form of a drive spring 140. The drive spring is provided in the form of a helical compression spring which in the state prior to actuation is maintained in an initial compressed state between an upper spring seat formed as a distal facing surface in the upper capsule part 110 and a lower spring seat formed by a proximally facing surface of a flange 155 arranged at a distal portion of ram 150. In the initial pre-firing state, the drive spring 140 is arranged coaxially with the ram 150 so that the drive spring partly surrounds a proximal tubular portion 154 of ram 150.

In example device 10, prior to firing of the capsule device 10, the drive spring 140 is held in the initial compressed state by means of a disc 160 which serves as a dissolvable firing member that provides a holding force for maintaining the drive spring 140 in its compressed state. Disc 160 is arranged between a proximally facing disk mounting surface, arranged in the lower capsule part 120, and a distal facing surface of the flange 155 on ram 150. The disc 160 is made of a material that dissolves when subjected to a fluid, such as gastric fluid. The disc 160 thus releasably holds the drive spring 140 in its initial compressed state until the disc is sufficiently dissolved so that the force of drive spring 140 overcomes the holding force of the disc thereby releasing the drive spring. In the shown example, capsule device 10 include a plurality of openings 116 for introducing gastric fluid within the capsule to enable fluid interaction with disc 160.

In a self-righting capsule device, by having a design with the capsule device resting with the central axis located in the direction of gravity and wherein the geometric center for the capsule and the center of mass offset axially downwardly from the geometric center towards the device bottom 123, when the capsule device is supported by a supporting surface while being oriented so that the centre of mass is offset laterally from the geometric center the capsule device experiences an externally applied torque due to gravity. This torque acts to orient the capsule device with the first axis oriented along the direction of gravity to enable the tissue engaging surface 123 to interact with the lumen wall at the target location.

In the shown embodiment, due to the density distribution of the entire capsule device 10, and due to the outside shape of the device, the capsule device 10 will tend to orient itself with the central axis substantially perpendicular to the surface (e.g., a surface substantially orthogonal to the force of gravity, a surface of a tissue such as the wall of the gastrointestinal tract). Hence, the capsule device tends to orient relative to the direction of gravity so that the tissue engaging surface 123 faces vertically downward.

After ingestion of the capsule device 10 into the stomach, gastric fluid will enter into the capsule and start interacting with disc 160. After a pre-determined time has lapsed, the disc 160 will be sufficiently dissolved to cause the ram 150 to be thrust distally towards the exit hole 124. The movement of ram 150 will stop when the ram hits a distally facing ram stop surface 128 arranged internally in lower capsule part 120. In this state a major portion of the payload portion 130 has been pushed externally to exit hole 124 of capsule device 10, and due to the tissue engaging surface 123 facing downward in contact with the lower portion of the stomach wall, the payload portion 130 has been injected into mucosal tissue, after which the payload portion 130 disconnects from the payload interface portion 156 of ram 150, leaving the payload portion inserted into tissue.

The self-righting capsule 10 shown in FIG. 1 has a height of approximately 12.1 mm and a largest lateral dimension of approx. 10.0 mm. The center of mass of the non-deployed capsule is located 3.6 mm from the bottom surface 123. The capsule parts for the example capsule device 10 has been selected as polycaprolactone (PCL) for the upper part 110 and 316L stainless steel for the lower part 120.

For state of art capsules it has been suggested to configure the self-righting capsule 10 with such density distribution, and with such geometrical shape and surface properties, that the self-righting capsule rolls without slipping relative to the mucosal tissue when a torque due to gravity acts on the self-righting capsule.

However, in accordance with the present invention, a capsule device may be provided with a surface that ensures that the mode of interacting with the stomach wall during self-righting will be a rolling movement incurring slipping between the capsule surface and the surface of the supporting stomach wall. In the following a theoretical framework for analysing self-righting via gravity is presented. In the following the capsule device 10 of FIG. 1 denotes the example capsule 10 wherein the surface portions of the capsule are relatively high friction surfaces, whereas example capsule device 100 (first embodiment) denotes a capsule having similar overall design but having surface portions provided as low friction surfaces.

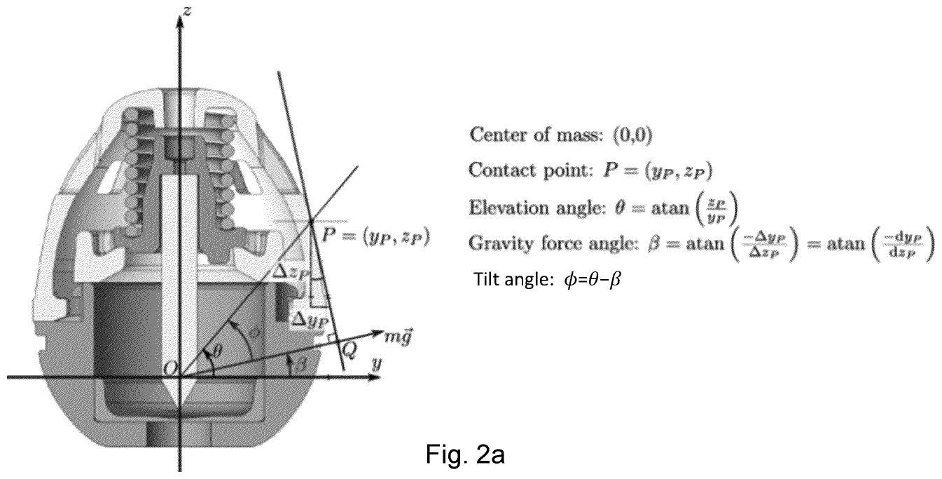

As an aid in investigating the ability of the capsule device 10 to self-orient relative to a supporting surface, FIGS. 2a and 2b show different geometric definitions for the capsule device shown in FIG. 1 with the device being exerted to a field of gravity and oriented for single point contact with a lumen wall in Contact point P. The center of mass of capsule device 10 is located at point O, with a coordinate system having its origin located at point O with the x-axis parallel with the central axis of the capsule device 10 (the firing axis) and with the y-axis pointing orthogonally to the central axis. The location of contact, the single point contact, between capsule 10 and the supporting surface is denoted P.

Elevation angle θ denotes the angle between the y-axis and a line extending between the Center of mass O and the contact point P. Angle β denotes the angle between the y-axis and the gravity, whereas the self-righting angle (tilt angle) is defined as ϕ=θ−β.

FIG. 2b show the above angles for a capsule device 10 in an example orientation and relative to the supporting surface. The supporting surface is arranged orthogonally to the field of gravity, the supporting surface thus representing a lower portion of the stomach wall of a patient. For capsule device 10 in the shown orientation, the center of mass O located laterally to the contact point P gives rise to a torque τ acting on the capsule device. For tilt angles ϕ larger than 0, the torque will be positive thus acting to orient the capsule device with the bottom surface 123 facing downwards.

FIG. 2c further show schematically four different example orientations of capsule device 10 relative to the supporting surface.

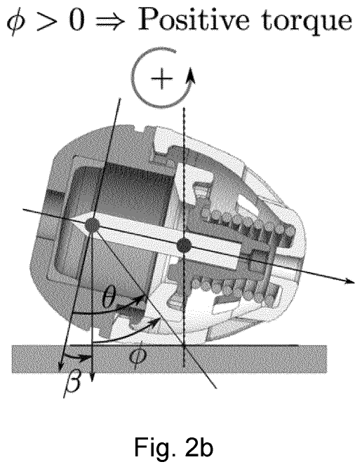

FIG. 3a shows the case wherein the capsule device rolls without slipping (friction) where the device rotates around point P_{c}, Pᵣ, and with the torque given as T =r_{⊥}mg.

FIG. 3b shows the case wherein the capsule device rolls with slipping (no friction) where the device rotates around point Pᵣ and with the torque given as τ=r_{⊥}mg. The torque arm for the two cases (friction and no friction) is of same magnitude, and hence the torque acting on the capsule device is the same.

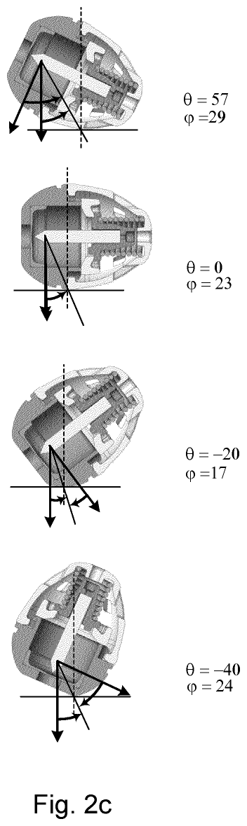

Next is considered the two cases wherein the capsule device, due to its weight, exerts surface pressure onto the soft mucosal tissue, causing the mucosal tissue to be pressed down relative to surrounding areas. This corresponds to a depression of the tissue, e.g. with a protrusion depth h such as in the order of a 0.2-0.6 mm. FIGS. 4a and 4b are representative for this condition, and it is seen that the torque arm r_{⊥}for the two cases (friction and no friction) differs markedly. Hence, for the two devices having the same shape and density distribution, the torque acting on device 10 (FIG. 4a) will be much smaller than the torque acting on device 100 (FIG. 4b). For both cases the outer contact points are denoted P_{c1} and P_{c2}.

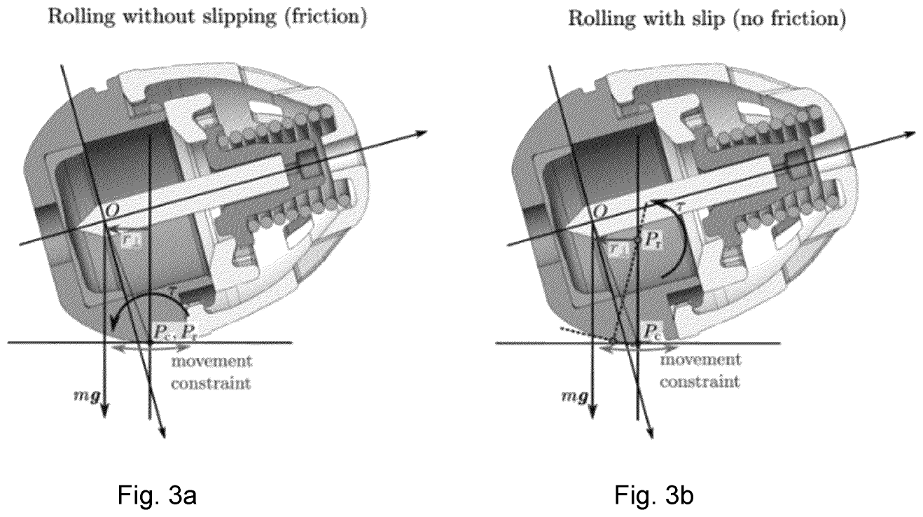

FIG. 5. shows different geometric definitions for the capsule devices 10, 100 and the equations used in calculating the torque arm and resulting torque for both the frictious case and the slippery case. Calculation for the torque can be done automatically in a programmed script that takes device shape, device mass, and device center of mass as inputs. With torque curves representing the torque acting on the capsule device due to gravity as a function of elevation angle for the capsule device, the critical surface potions of the capsule device, i.e. surface portions wherein surface properties for obtaining slippery movement will be beneficial, can be determined. Typically, a major portion of the external surface of the capsule device, such as the entire external capsule surface, may be provided as a low friction surface. However, in some embodiments, low friction surfaces are specifically provided at surface portions being in contact with the supporting tissue for elevation angles wherein the torque is found to be low, i.e. wherein the self-righting capability is comparatively low.

Any know methods for providing low-friction surfaces may be used in designing a low friction capsule, e.g. by utilizing surface polishing (surface roughness), surface geometry, surface micro-geometry and surface coating. A non-limiting example of providing a low coefficient of friction for the surface of the capsule device 100 may be or include a surface treatment, such as surface coatings known from medical implements, such as catheters for being introduced into a body lumen. Example surface coatings include those materials, coatings and compositions known from urinary catheters, e.g. as disclosed in WO 2019/034222 A1 and WO 98/58988 A1. Exemplary surface properties include surfaces having a coefficient of static friction in the order of 0.01-0.20, such as 0.01-0.1. For some embodiments, the coefficient of static friction is in the order 0.02-0.05. For a polished material for the capsule device exterior surface, exemplary surface finishes may be provided in the range Ra 0.02 to Ra 0.80.

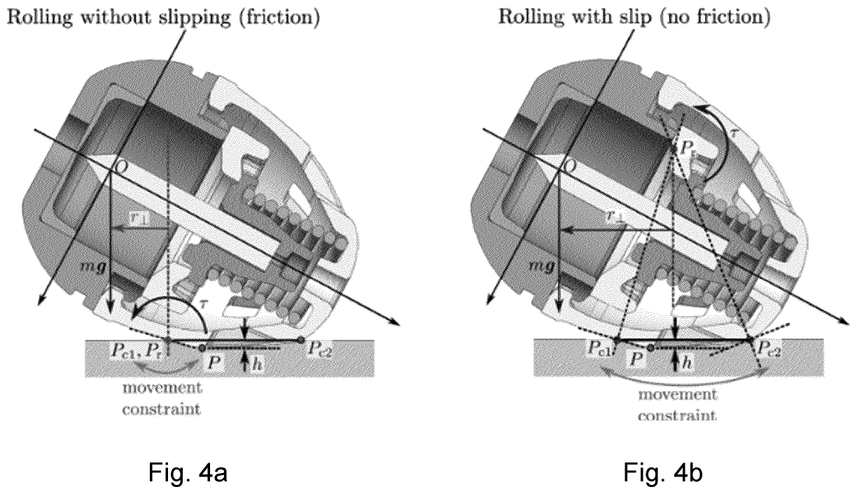

FIG. 6a shows a second embodiment of a capsule device 200 in accordance with the invention. The self-righting capsule 200 has a height of approximately 15.1 mm and a largest lateral dimension of approx. 12.0 mm. The center of mass of the non-deployed capsule is located 3.2 mm from the bottom surface 123, whereas the center of volume is disposed 6.8 mm from the bottom. The capsule parts for the second embodiment capsule device 200 has been selected as polycaprolactone (PCL) for the upper part 110 and 316L stainless steel for the lower part 120. The layout of capsule device 200 has been redesigned relative to capsule device 100 to provide a lower center of mass primarily by lowering the position of the disc 160. Also the material thickness of the capsule lower part 120 has been increased. The mass of capsule device 200 is 3.5 g.

In connection with FIGS. 6b and 6c, for the second embodiment capsule device 200, an analysis as to how the contact points and resulting torque changes as a function of protrusion depth has been made. FIG. 6b is a diagram for one particular elevation angle θ. The protrusion depth h denotes how far into the tissue the device will protrude. As h changes, so will the contact area between the device and the tissue. The near vertical lines in the diagram in FIG. 6b show the tissue surface for 0 mm, 0.2 mm, 0.4 mm and 0.6 mm protrusion depth, respectively. The stars denote the resulting center of rotation for each of these protrusion depths.

For each set of protrusion height, numerical calculations for all elevation angles have been made with the resulting torque provided as output. FIG. 6c show the results both for the slippery case (capsule device 200 with low friction surface) as well as for the frictious case (a capsule device similar to device 200 but with high friction surface). The dark line denotes protrusion height 0 mm corresponding to the singular contact case. It is readily apparent that the capsule device 200 is associated with a markedly higher self-righting torque exerted by gravity by all protrusion depths compared to the frictious case.

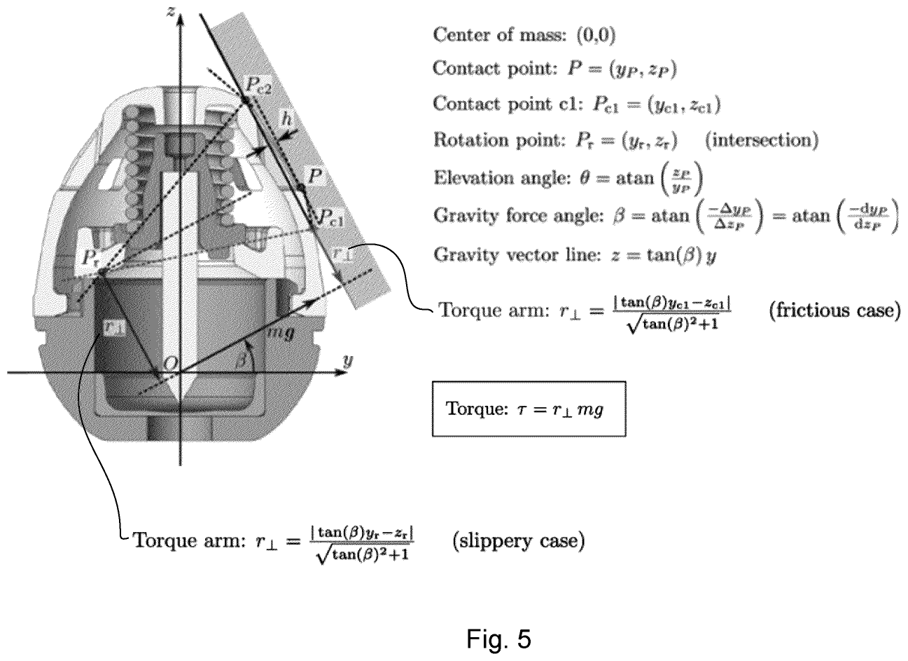

FIG. 7a shows a third embodiment of a capsule device 300 in accordance with the invention. The self-righting capsule 300 has a height of approximately 12.7 mm and a largest lateral dimension of approx. 12.0 mm. The center of mass of the non-deployed capsule is located 2.5 mm from the bottom surface 123, whereas the center of volume is disposed 5.8 mm from the bottom. The capsule parts for the second embodiment capsule device 300 has been selected as polyamide for the upper part 110 and 316L stainless steel for the lower part 120. The mass of capsule device 300 is 2.2 g. The layout of capsule device 300 has been redesigned relative to capsule devices 100 and 200 to provide a near spherical outer shape while still obtaining a low center of mass.

In connection with FIGS. 7b and 7c, for the third embodiment capsule device 300, an analysis as to how the contact points and resulting torque changes as a function of protrusion depth has been made. FIG. 7b is a diagram for one particular elevation angle θ. The protrusion depth h denotes how far into the tissue the device will protrude. As h changes, so will the contact area between the device and the tissue. The near vertical lines in the diagram in FIG. 7b show the tissue surface for 0 mm, 0.2 mm, 0.4 mm and 0.6 mm protrusion depth, respectively. The stars denote the resulting center of rotation for each of these protrusion depths.

Again, for each set of protrusion height, numerical calculations for all elevation angles have been made with the resulting torque provided as output. The results are shown in FIG. 7c, both for the slippery case (capsule device 300 with low friction surface) as well as for the frictious case (a capsule device similar to device 300 but with high friction surface). The dark line denotes protrusion height 0 mm corresponding to the singular contact case. Also for this case it is readily apparent that the capsule device 300, as compared to the frictious case, is associated with a markedly higher self-righting torque exerted by gravity by all protrusion depths.

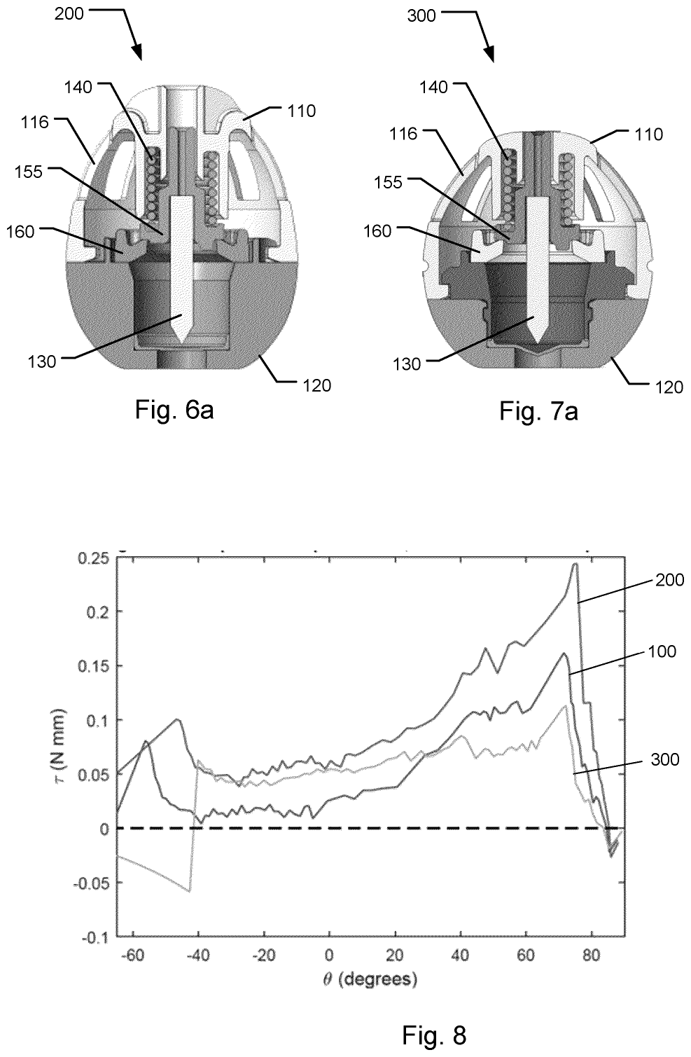

FIG. 8 shows a comparison of the above embodiments of self-righting capsule devices, i.e. first embodiment 100, second embodiment 200 and third embodiment 300 in the case of single contact point, i.e. with a protrusion depth of 0.0 mm. The capsule device 300 showed the best self-righting properties, even though it has the lowest device mass. Positive torque (τ>0) implies self-righting in the desired direction. The negative values for the capsule device 300 for angles θ<−40 deg. is due to a numerical error.

In particular from FIGS. 6c and 7c it is concluded that for the self-righting capsule devices 200 and 300 good self-righting properties are ensured for all orientations, provided that the exterior surface portions of the device that are in contact with tissue has a low value for the coefficient of static friction. This is naturally most relevant for elevation angles wherein the torque is deemed to be comparatively low, i.e. for the investigated embodiments, typically for lower angles of θ.

In order to estimate the critical friction coefficient where the self-righting capsule devices are hindered, due to friction, in self-righting by rotating about itself, the following examples are considered.

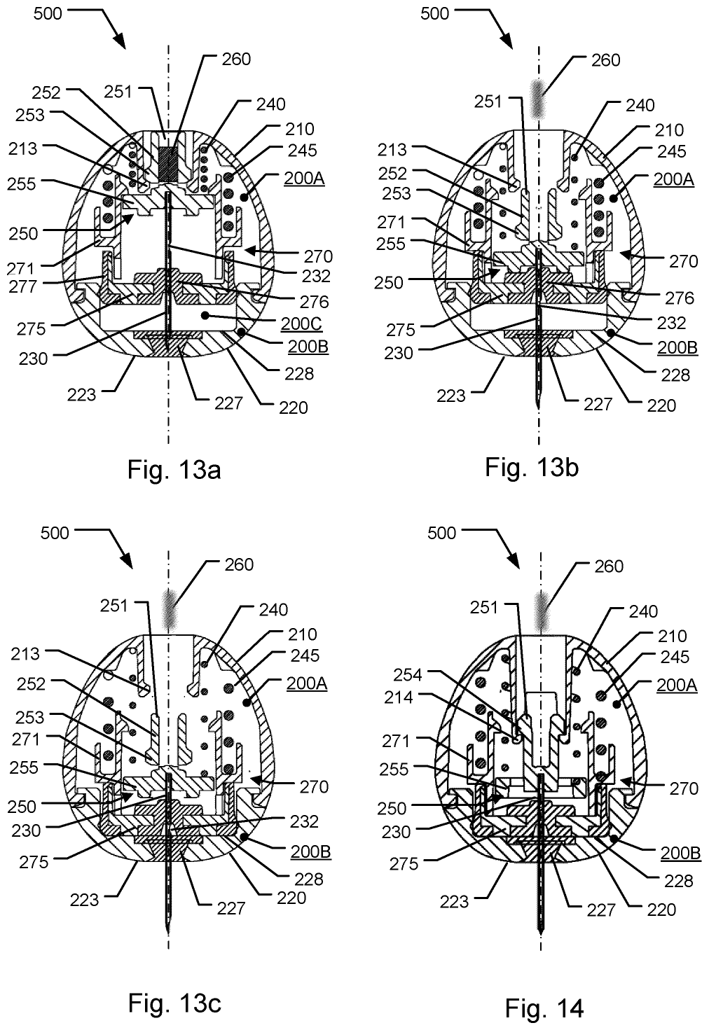

From FIGS. 6c and 7c it is noted that the self-righting torque generally seems to be lowest around a contact point elevation angle θ≈−30°, i.e. the most critical case. Based on this condition, the torques resulting from gravity and friction when the device is starting from rest in this position will be investigated. Reference is made to FIG. 15 which is a representation of the second embodiment self-righting capsule device 200 arranged in such angled start position. As it has been observed in numerous experiments, and predicted by theory, the device rotates about ‘itself’, i.e. the point of rotation is seen to be close to the center of volume. This is also the case where the contact point elevation angle is θ≈−30°. We therefore approximate the center of volume (see black cross in FIG. 15) to be the center of rotation for this analysis. Thus, to understand the self-righting, we study the resulting torques around this point.

Referring to FIG. 15, the variables involved are the following:
m—device mass [kg]
g—gravitational acceleration [m/s²]
d_{grav}—perpendicular (horizontal) distance from center of mass (grey cross) to the center of rotation (black cross) [mm]
h—Distance between center of mass (grey cross) and approximate center of rotation (black cross) [mm]
a—Angle between horizonal and the device centerline when the
contact angle is θ=−30° [°]
d_{fric}—perpendicular (vertical) distance from the lowest contact point to the center of rotation (black cross) [mm]

Forces:
Gravity force: F_{grav}=—mg e_{y} (acts at the center of mass)
Friction force: F_{fric}=—mgμeₓ (worst case: acts at the lowest contact point)

Torques (around the center of volume, marked by the black cross):
Gravity torque: τ_{grav}=—d_{grav} F_{grav}=h cos(α) mg
Friction force: τ_{fric}=d_{fric}F_{fric}=—d_{fric} mgμ

Total torque must be positive to ensure self-righting:

$${τ}_{tot} = {τ}_{grav} + {τ}_{fric} > 0 ⇒ μ < {μ}_{c} = \frac{h cos ( α )}{{d}_{fric}}$$

There are two forces of main concern: 1) the gravity force F_{grav} and 2) the friction forces F_{fric} between the device and the substrate it is resting on, see FIG. 15.

The gravity force is given by F_{grav}=—mg e_{y}, where m is the device mass and g is the gravitational acceleration, and it produces a positive torque τ_{grav}>0 around the center of rotation, tending to rotate the device counter-clockwise thus performing self-righting. The torque is identified by finding the horizontal distance (perpendicular to the gravity force) from the force's point of attack (center of mass, denoted by an grey cross) to the center of rotation d_{grav}, giving the torque:

$${τ}_{grav} = - {d}_{grav} {F}_{grav} = h cos ( α ) mg .$$

The friction forces will, if possible, resist this sliding motion between the device and the substrate. The normal forces on the SOMA device giving rise to friction forces are distributed over the contact area, but for our purposes, we consider the worst-case scenario: namely if the total friction force acts at the one singular point where it can generate to the biggest possible torque to resist the sliding motion. This is the point on the contact area that has the maximum vertical distance to the center of rotation, i.e. the lowest point of the contact, giving the distance d_{fric}. At this contact point, the summed friction force is F_{fric} =—mgμ eₓ, where ₁,1 is the static coefficient of friction between the device and the substrate, which produces a negative torque Tfᵣi_{c} <0, resisting torque in the counter clockwise direction (the friction itself will not rotate the device in the clockwise direction, it is always an opposed force). The arm for the friction torque is the vertical distance (perpendicular to the friction force) from the lowest contact point to the point of rotation d_{fric}. The friction torque becomes

$${τ}_{fric} = - {d}_{fric} {F}_{fric} = {d}_{fric} μ mg .$$

In the case where the friction forces is able to hinder self-righting, the friction torque exactly balances the gravity torque

$${τ}_{grav} + {τ}_{fric} = 0 at μ = {μ}_{c} .$$

Equating the two torques, and solving for the coefficient of friction, we find the critical coefficient of friction

$${μ}_{c} = \frac{h cos ( α )}{{d}_{fric}} ,$$

i.e. to ensure self-righting of the SOMA device, we have to ensure μ<μ_{c}, meaning that the desired coefficient of friction should be lower than critical friction coefficient μ_{c}.

In the following three cases are analyzed: 1) a simplified estimate from a spherical geometry, 2) an estimate for the second embodiment capsule device 200 discussed in connection with FIGS. 6a, 6b and 6c, and 3) an estimate for the third embodiment capsule device 300 discussed in connection with FIGS. 7a, 7b and 7c.

### EXAMPLE 1

### Simplified Spherical Device

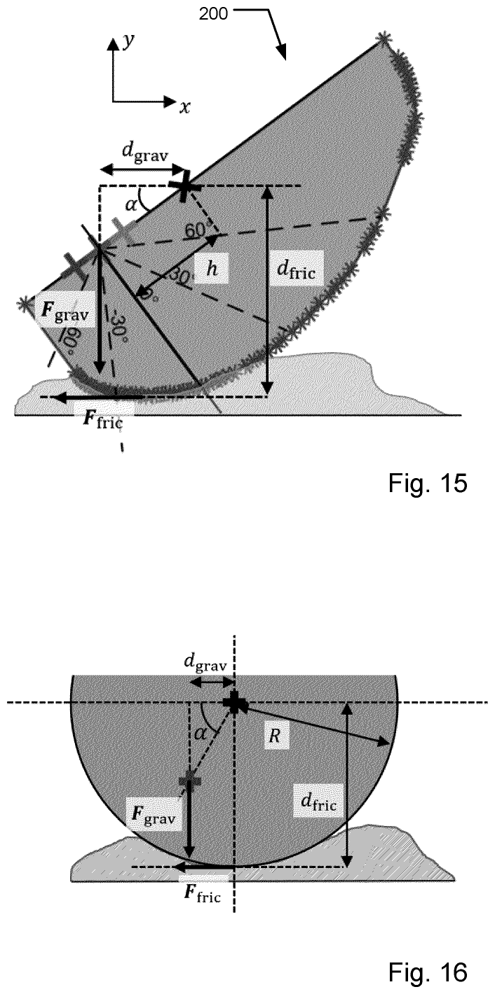

For this case, the device is considered locally to be spherical with radius R, see FIG. 16.

Forces
Gravity force: F_{grav}=—mg e_{y}
Friction force: F_{fric}=—,gμeₓ

Torques (around the center of volume, marked by the black cross)
Gravity torque: τ_{grav}=d_{grav} F_{grav}=1/2R cos(α) mg
Friction force: τ_{fric}=—d_{fric} F_{fric}=—R mgμ

Total torque must be positive to ensure self-righting:

$${τ}_{tot} = {τ}_{grav} + {τ}_{fric} > 0 ⇒ μ < \frac{h cos ( α )}{{d}_{fric}} ⇒ μ < \frac{\frac{1}{2} R cos ( α )}{R} = \frac{1}{2} cos ( α )$$

For the center of mass, we observe that many of the proposed self-orienting capsule devices have a center of mass similar (or lower even) to that of 1/2R from the center of mass, so here h=1/2R, and α=60°, and for the friction force we have d_{fric}=R. The critical coefficient becomes

$${μ}_{c} = \frac{\frac{1}{2} R cos ( 60 ° )}{R} = 0.25 .$$

Hence, based on these calculations, in order to ensure that the spherical device according to Example 1 perform slipping movement, the surface properties of the exterior surface touching the tissue should have a coefficient of friction below 0.25 and ideally even lower. It is to be noted that this result is derived from a simplified case.

### EXAMPLE 2

### Self-Righting Capsule Device, Second Embodiment

For the second embodiment self-righting capsule device 200 described in connection with FIGS. 6a, 6b and 6c we have the following conditions:
h=3.6 mm, α=42° and d_{fric}=7.3 mm

$$μ < \frac{3.6 mm cos ( 42 )}{7.3 mm} = 0.37 ⇒ {μ}_{c} = 0.37$$

Hence, based on this design, in order to ensure that the device according to Example 2 perform slipping movement, the surface properties of the exterior surface touching the tissue should have a coefficient of friction below 0.37 and ideally even lower.

### EXAMPLE 3

### Self-Righting Capsule Device, Third Embodiment

For the third embodiment self-righting capsule device 300 described in connection with FIGS. 7a, 7b and 7c we have the following conditions:
h=3.4 mm, α=56° and d_{fric}=6.6 mm

$$μ < \frac{3.4 mm cos ( 56 ° )}{6.6 mm} = 0.29 ⇒ {μ}_{c} = 0.29$$

Hence, based on this design, in order to ensure that the device according to Example 3 perform slipping movement, the surface properties of the exterior surface touching the tissue should have a coefficient of friction below 0.29 and ideally even lower.

### END OF EXAMPLES

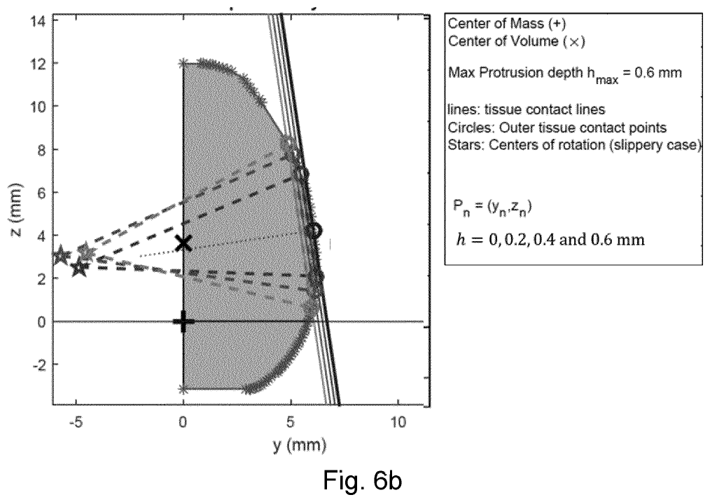

Turning now to FIGS. 9a and 9b a fourth embodiment of an ingestible self-righting capsule device 400 is shown. Device 400 comprises a first portion 100A having an average density, a second portion 100B having an average density different from the average density of the first portion 100A. The capsule device 400 accommodates a payload portion 130 for carrying an agent for release internally of a subject user that ingests the article. In the shown embodiment, the average density of capsule device prior to deployment is larger than that of gastrointestinal fluid, enabling the capsule device to sink to the bottom of the stomach lumen. The outer shape of the self-righting article is a gomboc shape, i.e. a gomboc-type shape that, when placed on a surface in any orientation other than a single stable orientation of the shape, then the shape will tend to reorient to its single stable orientation. Also for this embodiment superior self-righting capability has been obtained by using a capsule outer surface with low friction.

The capsule device shown includes an upper (proximal) capsule part 110 which mates and attaches to a lower (distal) capsule part 120. The upper capsule part 110 and the lower capsule part 120 together forms the capsule housing of the device. The capsule defines an interior hollow which accommodates the payload portion 130, a ram 150 which holds and drives forward the payload portion 130, and a firing and propulsion mechanism including an actuator configured to fire and drive forward the ram with the payload for drug delivery. The payload portion 130 is oriented along a firing axis and configured for movement along the firing axis. In the shown embodiment, the upper and lower capsule parts 110, 120 form rotation symmetric parts which are symmetric around the firing axis. In FIG. 1, the device 10 is oriented with the firing axis pointing vertically, and with the payload portion 130 pointing vertically downwards towards an exit hole 124 arranged centrally in the lower capsule part 120, the exit hole allowing the payload portion 130 to be transported through exit hole and moved outside the capsule device 400. The lower part 120 includes a tissue engaging surface 123 which is formed as a substantially flat lower outer surface surrounding the exit hole 124.

Regarding suitable materials for the capsule parts for the embodiment shown in FIGS. 9a and 9b, the upper part may suitably be made from a low-density material, such as polycaprolactone (PCL), whereas the lower part 120 may be suitably made from a high-density material, such as 316L stainless steel.

In the shown embodiment, due to the density distribution of the entire capsule device 400, and due to the outside shape of the device, the capsule device 400 will tend to orient itself with the firing axis substantially perpendicular to the surface (e.g., a surface substantially orthogonal to the force of gravity, a surface of a tissue such as the wall of the gastrointestinal tract). Hence, the capsule device tends to orient relative to the direction of gravity so that the tissue engaging surface 123 faces vertically downward.

The interior of the upper capsule 110 includes a sleeve shaped ram guiding structure 115 which extends concentrically with the firing axis from the upper part of the upper capsule part 110 towards a ram stop surface 128 defined by an inner bottom surface formed in the lower capsule part 120, i.e. a proximally facing stop surface. Further, in the shown embodiment, a second sleeve shaped structure extends concentrically with the firing axis and radially inside the ram guiding structure 115 from the upper capsule part 110 and downwards along the firing axis. The second sleeve shaped structure serves as a retainer structure for retaining the ram 150 against the drive force emanating from a strained drive spring 140 arranged within the capsule, i.e. the drive spring serves as an actuator for driving forward the ram from a first position to a second position. In the shown embodiment, the retainer structure has a radially inwards protruding retainer portion 113 arranged at the lower end of the retainer structure. In the shown embodiment, the retainer portion 113 is provided as two opposed radially inwards protruding arc-shaped protrusions.

In the fourth embodiment shown in FIGS. 9a and 9b, payload portion 130 defines a solid delivery member formed entirely or partly from a preparation comprising the therapeutic payload. In the shown embodiment, the solid delivery member is formed as a thin cylindrical rod shaped to penetrate tissue of the lumen wall, the cylindrical rod having a tissue penetrating end and trailing end opposite the tissue penetrating end. The tissue penetrating end of the rod is pointed to facilitate easy insertion into tissue of the lumen wall whereas the trailing end, in the shown embodiment, defines a truncated cylinder cut off by a 90-degree cut. A non-limiting example of a drug suitable for delivery by capsule device 400 is dried compressed API such as insulin.

The ram 150 comprises an upper retaining part 151 and a lower interface part 155 configured for holding the trailing end of the payload portion 130 in place. In the shown embodiment, the interface part includes a downward open bore that receives the trailing end of the payload portion 130 in a way so that the payload portion 130 is firmly attached within the bore. The lower interface part 155 further defines an annular outer flange having a diameter slightly smaller than the diameter of the ram guiding structure 115. In the shown embodiment, the ram 150 is movable, while being guided for axial movement by the ram guiding structure 115, from a pre-firing configuration shown in FIG. 9a to a firing configuration shown in FIG. 9b.

With regard to the above-mentioned drive spring 140, in capsule device 400, a helical compression spring is arranged coaxially with the firing axis. The proximal end of drive spring 140 is seated against a spring seat of upper capsule part 110, i.e. located radially between the ram guiding structure 115 and the retainer structure. The distal end of drive spring 140 is seated against a spring seat formed by a proximal surface of the flange defined by the lower interface part 155 of the ram 150. As part of assembling the capsule device 400 the drive spring 140 has been energized by axially compressing the drive spring 140 between the two spring seats. Hence, the ram is initially under load from drive spring, such as in the order of 10-30 N. Alternatives to using a compression spring for generating the drive force, other spring configurations may be used to energize the capsule device 400, such as a torsion spring, a leaf spring, a constant-force spring or similar. In further alternatives, a gas spring or a gas generator may be used.

The upper retaining part 151 of the ram 150 includes deflectable latches provided in the form of two deflectable arms 152 which extend in distal direction from the upper end of the ram towards the exit opening 124, each arm being resiliently deflectable in the radial inwards direction. The end of each deflectable arm 152 includes a blocking portion 153 protruding radially outwards from the resilient arm. In the pre-firing configuration shown in FIG. 1a, a distal surface of each of the blocking portions 153 engage a proximal surface of a corresponding one the retainer portions 113. l As the blocking portions 153 initially are located proximally to the retainer portions 113 the ram 150 cannot be moved distally past the retainer portions 113 unless the deflectable arms 152 become sufficiently deflected in the radially inwards direction.

In the pre-firing configuration a dissolvable pellet 160 is arranged between the two deflectable arms 152 so that radial opposing surfaces of the pellet 160 engage a radially inwards facing support surface of the two deflectable arms 152. In the shown embodiment, the pellet 160 is arranged in a compartment inside the upper capsule part 110, and a proximally arranged upper opening in upper capsule part 110 facilitates fluid exposure to the dissolvable pellet when the capsule device is submerged in a fluid. In the pre-firing configuration shown in FIG. 1a, as the dissolvable pellet 160 assumes a non-compressle state the pellet prevents the two deflectable arms from bending inwards. However, upon exposure to a fluid, such as gastric fluid present in the stomach of a patient, the dissolvable pellet starts to dissolve. The pellet 160 is designed to become gradually dissolved so that after a predefined activation time, the pellet has been dissolved to a degree allowing the two deflectable arms 152 to become sufficiently deflected inwards enabling the blocking portions 153 of ram 150 to be moved distally past the retainer portions 113. In this condition, i.e. the firing configuration, the ram 150 has been fired with the load of the drive spring 140 forcing the ram 150 distally towards the exit hole 124. The ram 150 drives the payload portion 130 distally with the payload tip protruding initially from the capsule, and gradually pressing out the remaining payload portion 130. The forward movement of the payload portion 130 is halted when ram 150 bottoms out in the lower capsule part 120. This condition is depicted in FIG. 1b.

In the shown embodim, the interface between the retainer portions 113 and the blocking portions 153 is sloped by approximately 30° so that the deflectable arms will slide inwards when the dissolvable pellet is dissolved. The angle determines the shear forces on the pellet and to which degree the deflectable arms will tend to slide inwards when subjected to the load force. In connecn with the acceleration length of the ram when fired, the optimal angle is 0°, but it requires a much higher spring force to activate such configuration. For the sloped portions, in other embodiments, angles other than 30° may be used.

FIG. 9b reveals that, in the shown embodiment, the ram 150 and the payload portion 130 may enter an orientation that is somewhat tilted relative the firing axis. This effect is obtained by a tilting mechanism that tilts the ram 150 upon the ram reaching its end destination. However, this shown condition is somewhat hypothetical, as it is only representative for a capsule device being fired into the open, or with the payload portion being fired into a fluid.

In situation of intended use, the payload portion 130 is inserted into tissue of the lumen wall where it will anchor generally in a direction along the firing axis. However, at the end of the drive stroke, and due to the tilting action of the ram 150, a bending torque is applied onto payload portion 130 tending to break or otherwise release the connection between payload 130 and ram 150. This effect is introduced to enable the payload portion 130 to become forcedly separated from the ram 150 to prevent that payload portion 130 becomes withdrawn from the tissue after it has been properly lodged within the tissue.

At this point the capsule device 400 has delivered the intended dose and will release relative to the deposited payload portion 130 which rests inside the tissue wall. Subsequently, the remaining parts of the capsule device will travel out through the digestive system of the user and be disposed of.

If the payload 130 where still fixedly connected to ram 150, and thus also to the remaining parts of the capsule device 400, the likelihood that payload portion would become retracted from the tissue by movements of the capsule device relative to the target location would be high.

In the shown embodiment, the tilting motion of ram 150 upon reaching the end destination is obtained by forming an eccentrically arranged protrusion 158 on the distally facing surface of interface part 155 of ram 150. As proximally facing ram stop surface 128 defined by the inner bottom surface formed in the lower capsule part 120 is planar, and oriented orthogonally to the firing axis, a tilting effect is obtained asam 150, meets the ram stop surface 128. As will be discussed further below, the tilting effect may be obtained by a variety of alternative geometrical designs. Also, although not depicted in this disclosure, a guide system between ram guiding structure 115 and the ram 150 may alternatively be formed to obtain a similar tilting effect. It should also be noticed that in other embodiments of a capsule device, the tilting effect may be omitted.

For the dissolvable member discussed above, i.e. the dissolvable pellet 160 forming a dissolvable firing member, different forms and compositions may be used. Non-limiting examples include injection moulded Isomalt pellets, compressed granulate Isomalt pellets, compressed pellets made from a granulate composition of Citrate/NaHCO3, or compressed pellets made from a granulate composition of Isomalt/Citrate/NaHCO3. A non-limiting exemplary size of a dissolvable pellet is a pellet which at the time of manufacturing measures Ø1×3 mm.

In the shown example of ram 150 the upper retaining part 151 is formed as a chamber wherein the dissolvable pellet 160 is received within the chamber having a tight fit. In the shown embodiment, the central upper part of capsule device 400 includes a single opening for introducing stomach fluid within the capsule. In other embodiments, the capsule may include other designs of fluid inlet openings such as multiple openings distributed around the capsule. In some designs, the payload portion 130 is accommodated in a chamber that is fluidly sealed from the chamber of the dissolvable pellet. Also, the exit hole 124 may include a seal preventing moisture from entering the payload portion chamber prior to firing of the capsule device 400.

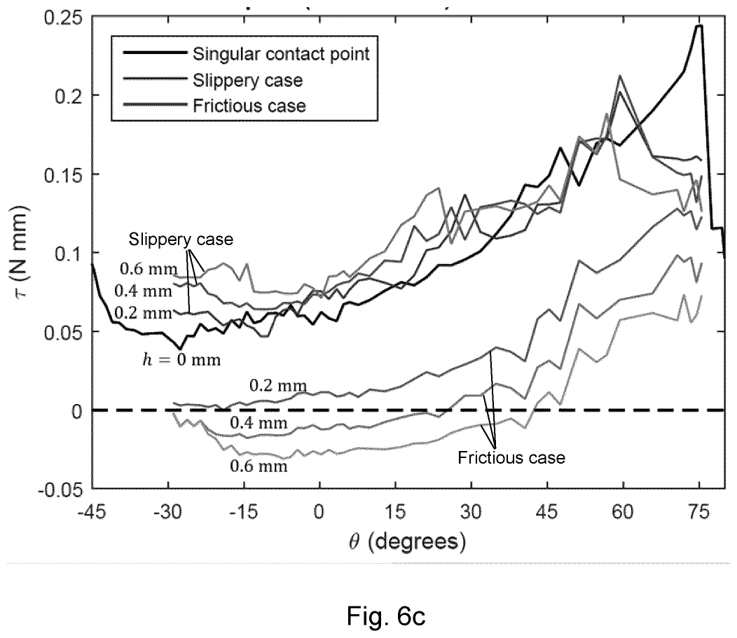

Turning now to FIG. 10, three alternative suitable designs for a ram and payload portion are schematically depicted, each design obtaining a desired attachment between ram 150 and payload portion 130 and enabling a desired controlled detachment of payload portion 130 from ram 150.

Design no. I includes a ram 150 having a central pin 156.I extending from lower interface part 155 of the ram 150. Payload portion 130 is correspondingly formed with a central opening configured for receiving central pin 156.II.

Design no. II includes a ram 150 having a central conical protrusion 156.II extending from lower interface part 155 of the ram 150. Payload portion 130 is correspondingly formed with a central conical depression configured for mating with and receiving conical protrusion 156.II.

Design no. III includes a ram 150 having a central conical depression 156.III at the distal facing surface of lower interface part 155 of ram 150. Payload portion 130 is correspondingly formed with a central conical protrusion configured for mating with and receiving conical protrusion 156.III.

The above described different variants of payload interface portions 156 between the payload portion 130 and the ram 150 are only exemplary and other configurations may be used instead. The detachable attachment between the payload portion and the ram may be obtained by using a friction or press fit. Alternatively, an adhesive may be used at the interface, such as sucrose. Still alternatively, the attachment may be obtained by initially wetting the payload portion and utilizing inherent stiction between the ram and the payload portion. In situation of use, upon the ram reaching its final destination, detachment may occur at the interface between the payload portion and the ram. In other embodiments, a desired detachment may be obtained by detaching a major portion of the payload portion from the remaining payload portion being still adhered or fastened to the ram. In some embodiments, the payload portion includes a weakened point which determines the point of separation. In still further embodiments, as discussed further below, the ram and the payload portion may be formed as a unitary component all made of a composition containing API, and wherein the intended payload portion to be pushed out from capsule device is separated from the ram portion. Also, in alternative embodiments, the payload may act as a ram by itself to be fully transported away from the capsule device.

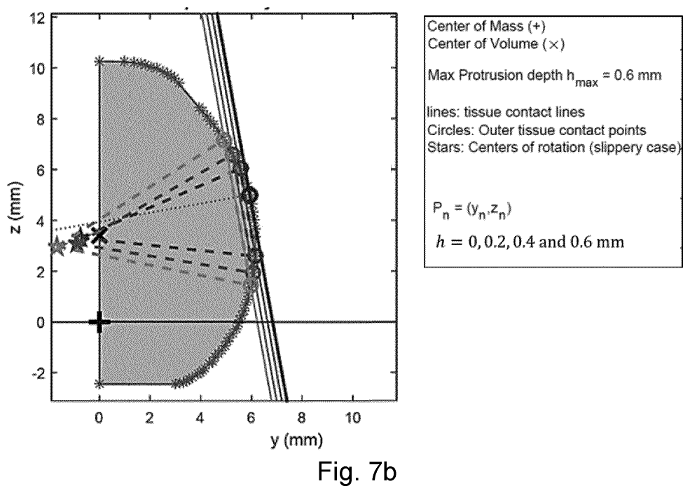

FIG. 11 schematically shows four additional designs for one or two pairs of deflectable latch and retainer configurations to be used in further exemplary capsule devices. As will be readily apparent, the number of deflectable latch elements, the location and the orientation of deflectable latch elements, the number and configuration of dissolvable firing members as well as the design of the ram may be varied in agreement with an aspect of the present invention while still obtaining firing mechanisms having a superior mode of action. For simplicity, only the upper retaining part 151 of ram 150 has been shown. Likewise, only the retainer structure of the capsule parts have been shown.

In FIG. 11, design no. I a retainer portion having upwardly extending retaining structure 113 to cooperate with blocking elements on two deflectable arms 152 is shown. In this design, a ram and a dissolvable firing member 160 having an overall structure as shown in FIG. 9a may be used.

Design no. II also includes an upwardly extending retaining structure 113 wherein a major portion of the ram is suspended. In this embodiment, the ram includes proximally extending delectable arms having blocking elements on the proximal ends of the deflectable arms 152, and wherein the proximal ends of the arms are designed to flex radially inwards when a centrally located dissolvable firing member 160 is sufficiently dissolved.

The figure depicting design no. III shows a related configuration but wherein the ram only includes a single deflectable arm. In this design a non-deflectable structure is arranged on the side of the dissolvable firing member 160 on the side facing away from the single deflectable arm. The non-deflectable structure continuously supports the dissolvable firing member 160 on one side thereof whereas the opposing side makes room for the single deflectable latch arm to move radially inwards and pass the retainer portion 113.

Finally, design no. IV schematically shows an example wherein the deflectable latch and the retainer portions have swapped places. In this design the ram includes an upper retaining portion 151′ with retainer portions 153′ which are designed not to exhibit any flexure during firing of the actuation mechanism. The retaining structure (associated with either the upper capsule part or the lower capsule part) instead includes two deflectable latches in the form of distally extending deflectable latch arms 112′, each having a blocking portion 153′ at its most distal end. Each deflectable arm 112′ is configured to engage a respective dissolvable firing member 160′. Said respective dissolvable firing members 160′ may thus be provided as a common ring-shaped member or be provided as a plurality of separate members arranged in a ring-configuration around the firing axis. As noted above, in some embodiments, the payload may act as a ram by itself to be partly or fully disconnected from the remainder of the capsule device. Such API based ram may include retainer portions which are designed not to exhibit any flexure during firing of the actuation mechanism where the retainer portions are allowed to pass cooperating deflectable latches associated with the housing of the capsule, e.g. the upper or lower capsule parts.

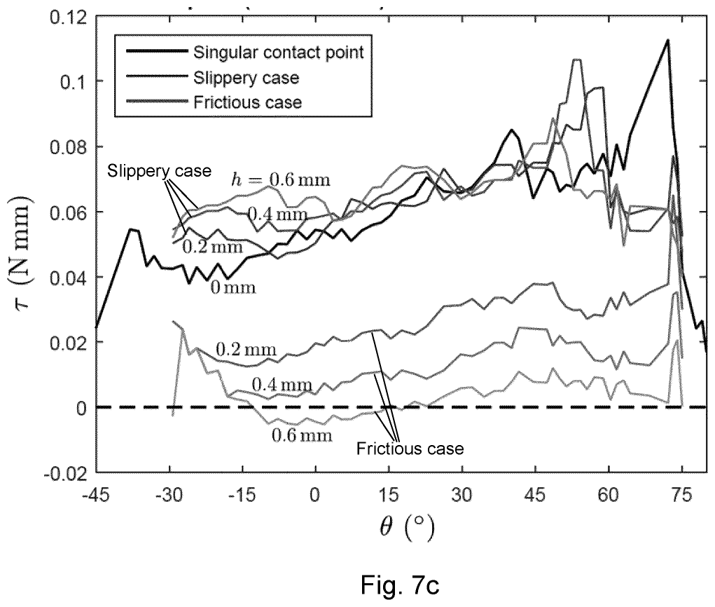

FIG. 12 schematically shows three designs for obtaining the tilting effect of the ram 150 as described above. In design no. I, an eccentrically disposed protrusion 158 is formed on the distally facing surface of interface part 155 of ram 150, i.e. the surface facing the ram stop surface 128. In design no. II, an eccentrically disposed protrusion 129 on the ram stop surface 128 is located to protrude in the proximal direction towards the lower surface of the interface part 155 of ram 150. In the variant shown as design III the ram stop surface 128 is formed as a stepped surface 129′, i.e. comprising two or more levels that induces a tilting movement of ram 150 as it reaches the ram stop surface 128. It is to be noted that other ways of tilting the ram upon reaching the final destination than shown schematically in FIG. 12 may be carried out by other means.

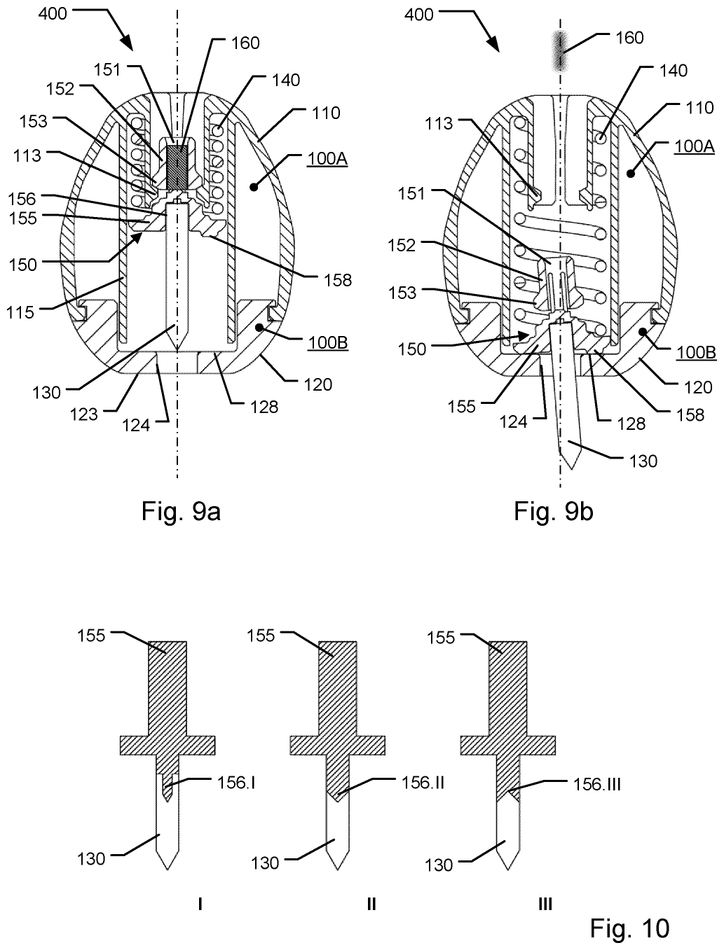

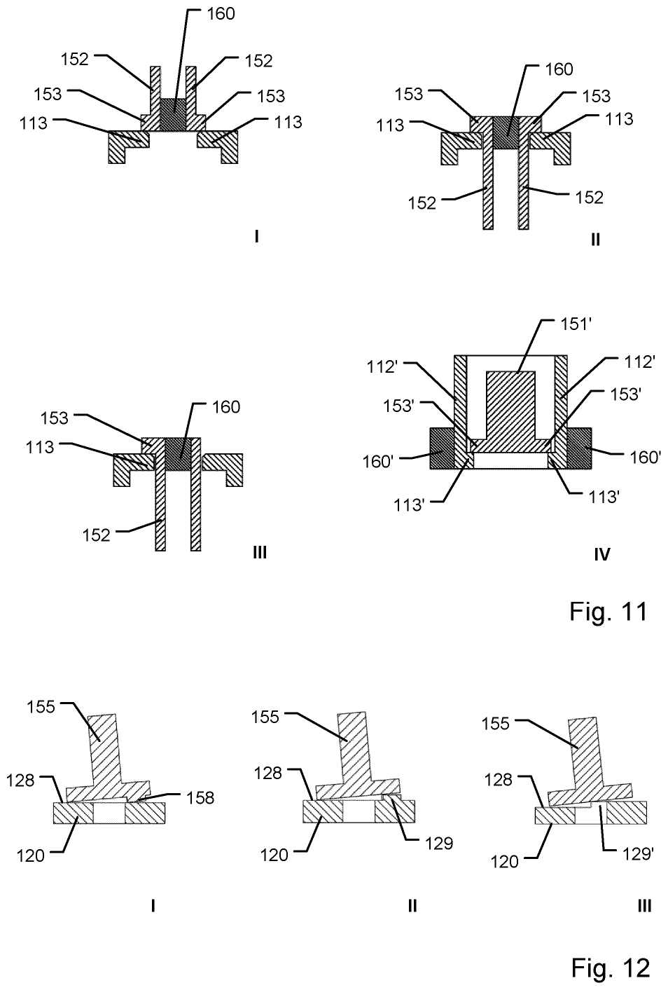

With reference to FIGS. 13a-13c and 14, a fifth embodiment of a drug delivery device in accordance with an aspect of the invention will next be described, the fifth embodiment being designed to provide a capsule device 500 having a desired firing principle for injection of dose of a liquid formulation from a liquid based capsule device. The disclosed embodiment relates to a capsule device 500 suitable for being ingested by a patient to allow the capsule device to enter the stomach lumen, to orient relative to the stomach wall, subsequently to deploy an injection needle for needle insertion at a target location in tissue of the stomach wall, and finally expel liquid through the injection needle. Similar to the first through forth embodiments, the fifth embodiment utilizes a capsule outer surface with low friction to provide superior self-righting capability.

The capsule device 500 comprises a chamber 200C for holding the liquid formulation prior to release in the gastrointestinal tract (e.g., in the stomach such as at the stomach wall); a needle-based delivery mechanism of the liquid; and a system for actuation of needle insertion and subsequent liquid expelling.

FIG. 13a-13c illustrate various exemplary components in a liquid based capsule device in three states during firing and performing an injection. Prior to injection, the liquid drug formulation is kept and protected inside the system by means of the chamber 200C with a volume of approximately 80 pL. This chamber 200C comprises three members that together makes a fully sealed inner volume; 1) a lower capsule 220 bottom portion, 2) an outer septum (e.g., plug) 227 made from silicone or TPE, 3) a plunger 275 provided as a 2K molded component made from a hard polymer and a soft TPE acting both as an inner septum 276 and an outer plunger seal 277. These septa are generally capable of sealing around the injection needle as well as preventing food or liquid from passing through from the outside environment. Therefore, for example, the enzymes in the stomach would not be able to reach the drug formulation through the septum, and the formulation would not leak out of the septum.

In order to deliver the liquid drug formulation into the tissue, an injection needle 230 is used to aid in delivery. The needle 230 is inserted directly through the inner septum 276, creating a tight-fitting seal. The needle is hollow (e.g., comprising a channel); however, the liquid formulation is not passed through the top of the needle. Instead, a hole (e.g., inlet) 232 is present in the side of the injection needle 230. Liquid is configured to pass through this hole and out of the beveled end, i.e. at the distal end of the injection needle. For example, the liquid chamber 200C (e.g., reservoir) may be placed in fluidic communication with the hole 232 upon activation of the spring 240, thus facilitating the transfer of fluid from the liquid chamber 200C into the needle. The hole 232 is located at a height on the needle such that the hole is outside the liquid chamber 200C prior to activation, i.e. as shown in FIG. 13a. When the device is actuated, in this example, the needle is moved e.g., 5 mm down. As shown in FIG. 13b, this movement inserts the needle 230 into the stomach tissue as well as moving the side hole 232 into the liquid chamber 200C enabling a flow path from the chamber to the tissue. The top end of the needle may be closed off and used as a connection point to actuating spring 240 via a needle hub 255. Therefore, the only way for fluid to move through the needle 230 is from the hole 232 in the side to the hole in the tip.

The capsule device 500 autonomously orients in the stomach after ingestion in order to align its injection mechanism with the tissue in the same manner as in the first through fourth embodiments. The device's high curvature upper portion coupled with its low center of mass ensure that it only possesses one stable orientation, defined as an angle in which the device's center of mass is at a local minimum. Additionally, the flattened bottom of the capsule device 500 stabilizes its preferred configuration and ensures that it does not tip over and misfire into the lumen if a patient moves about during actuation. The firing mechanism of capsule device 500 generally correspond to the firing mechanism of capsule device 400 according to the fourth embodiment.

The capsule device 500 includes a releasable firing mechanism incorporating a dissolvable firing member 206 generally similar to the capsule device 400 discussed above. As soon as the capsule device 500 is ingested, a hydration based actuator plug (e.g., made from isomalt) 260 begins to dissolve. The plug holds hub 255 connected to the injection needle 230 in place by means of two opposed deflectable arms 252 each having a blocking portion 253 engaging a proximal surface of a corresponding retainer portion 213. Once dissolved, the plug 260 releases the hub 255 and the compressed spring 240 expands to insert the needle 230 into the tissue. After a set distance, a stopping geometry 254 on the hub 255 is stopped by a tab 214 on the housing of the device (see FIG. 14). This ensures that the needle 230 inserts a set distance into the tissue.

Once the device inserts the needle, the needle hub 255 immediately actuates a second com pressed spring 245 which delivers the loaded liquid formulation by movement of plunger 275.

An intermediate driving component 270 is arranged between the second compressed spring 245 and the plunger 275 and provides both as an axial guide for the needle hub 255, as a spring seat 271 for the second compression spring 245, and as a driving member for transferring force from the second compression spring 245 onto the plunger 275. The plunger 275 bottoms out in the liquid chamber 200C when the plunger 275 engages the bottom surface 228 of liquid chamber 200C. For the shown embodiment capsule device 500, by decoupling the needle insertion from the liquid injection, the device is able to inject its entire liquid dose at an exact tissue depth instead of injecting the dose as the needle moves through the tissue.

The needle that is inserted into the tissue can either be removed from the tissue and brought back into the device via a retractable mechanism, a swelling hygrogel, or it can lose its sharpness. A third spring can be used to bring the needle back from its inserted state into the device. A dissolvable needle can be used to eliminate the needle. However, because the design currently uses a needle in contact with the fluid inside of the device, in some cases it would be desirable that it not dissolve from the outside surface. Therefore, for example, it may comprise a protective coating on the outside surface of the needle. Such a coating could be a metal such as gold or it could be a polymer such as parylene. This layer could be anywhere between 300 nm to 5 um thick. It is desirable that a dissolvable needle maintains its functionality after being inserted into the tissue. For example, it should be able to easily penetrate the tissue. In some cases it may use a relatively sharp tip. It may also be configured to pass liquid through an inside tube. Additionally, it may be configured to have a hole on the top section to allow liquid to enter. Some examples of materials that the needle could be made of include: a sugar or sugar like material such as isomalt or sucrose; a biodegradable polymer or co-polymer such as PVP, PVA, Soluplus; a hydrogel; gelatin; a starch. The needle may be configured to dissolve from the inside tube to the outside. If the needle hydrates and becomes soft, then this may also eliminate the potential for a perforation from the protruding needle. If there is a soft boundary made around the tip of the needle, then this may also prevent perforation. If the needle became floppy, such as a piece of pasta, then this may also work. If the needle broke into small pieces then this may also work. The needle could be made of a degradable metal, such that it would break up. Such metals include zinc, magnesium and iron along with others. In accordance with the delivery member separation principle as discussed above in connection with the fourth embodiment, and in connection with FIG. 12, a similar mechanism may be used for separation of the needle in the second embodiment.

Although the above description of exemplary embodiments for self-righting capsules mainly relate to ingestible capsules for delivery in the stomach, the present self-righting principle generally finds utility in capsule devices for lumen insertion in general, wherein a capsule device is positioned into a body lumen, and wherein the capsule device self-orients relative to a supporting lumen wall. The capsules may be configured for being ingested, or to be inserted into a body lumen by other routes than oral ingestion. Non-limiting examples of capsule devices may include capsule devices for intestinal delivery of a drug into the tissue wall of an intestinal lumen. Drug delivery may be performed using a delivery member, such as a needle, via micro-needles which is inserted into the tissue wall of a lumen. Alternative to using a dedicated delivery member, capsule devices which fires directly into the lumen wall, such as performed through one or more exit openings of the capsule device without the use of a delivery member, may be used. Exemplary embodiments for such devices include capsule devices which deliver one or more drugs by jet action wherein particles are introduced into the tissue wall by accelerating the particles against the lumen wall.

Alternatively, or in combination with drug delivery, the present self-righting principle generally finds utility in capsule devices for lumen insertion wherein one or more diagnostic actions is/are provided by the capsule device, Examples include the incorporation of sensing devices, such as sensors measuring a physical parameter, or sensing devices utilizing image sensing. Also, the use of one or more anchoring mechanisms may be incorporated with the self-righting capsule device, so that once the self-righting capsule has entered into a desired orientation relative to a lumen wall, an anchoring mechanism becomes deployed for maintaining the assumed orientation for a prolonged period of time.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A capsule device suitable for insertion into a lumen of a patient, the lumen having a lumen wall, wherein the capsule device comprises:
a capsule housing having an outside shape formed as a rounded object and defining an exterior surface, and
a tissue interfacing component disposed relative to the capsule housing, the tissue interfacing component configured to interact with the lumen wall at a target location,
wherein the capsule device is configured as a self-righting capsule having a geometric center and a center of mass offset from the geometric center along a first axis, wherein when the capsule device is supported by the tissue of the lumen wall while being oriented so that the centre of mass is offset laterally from the geometric center the capsule device experiences an externally applied torque due to gravity acting to orient the capsule device with the first axis oriented along the direction of gravity to enable the tissue interfacing component to interact with the lumen wall at the target location,
wherein at least a portion of the exterior surface of the capsule device has a surface property exhibiting one or more surface properties selected from the group consisting of a surface coating, a surface roughness, a surface geometry, and a surface micro-geometry, and
wherein said surface property is selected to provide low friction, such as low static friction, ensuring slipping movement of the capsule device relative to the tissue of the lumen wall when said externally applied torque due to gravity acts on the capsule device.

2. The capsule device as in claim 1, wherein said surface property is so selected that, when the capsule device is supported on a level surface, the low static friction ensures slipping movement of the capsule device relative to the tissue of the lumen wall when said externally applied torque due to gravity acts on the capsule device.

3. The capsule device as in claim 1, wherein the entire capsule exterior has said surface property.

4. The capsule device as in claim 1, wherein a lower part of the capsule device adjacent the tissue interfacing component, such as the lower half surface area of the capsule exterior surface area, includes surface portions having said surface property.

5. The capsule device as in claim 1, wherein the tissue interfacing component comprises a therapeutic payload configured to provide release of at least a part of the therapeutic payload to the lumen wall at the target location.

6. The capsule device as in claim 5, wherein the therapeutic payload is disposable or disposed in the capsule device, the therapeutic payload configured for being expelled from the capsule into the lumen wall at the target location.

7. The capsule device as in claim 5, wherein the tissue interfacing component comprises a delivery member disposable or disposed in the capsule device, the delivery member being shaped to penetrate tissue of the lumen wall and having a tissue penetrating end and a trailing end opposite the tissue penetrating end, the delivery member comprising the therapeutic payload or being configured to deliver the therapeutic payload from a reservoir.

8. The capsule device as in claim 7, wherein the capsule device further comprises an actuator coupled to the delivery member and having a first configuration and a second configuration, the delivery member being retained within the capsule housing when the actuator is in the first configuration, wherein the delivery member is configured to be advanced from the capsule housing and into the lumen wall by movement of the actuator from the first configuration to the second configuration.

9. The capsule device as in claim 7, wherein the delivery member is a solid formed entirely from a preparation comprising the therapeutic payload, wherein the delivery member is made from a dissolvable material that dissolves when inserted into tissue of the lumen wall to deliver at least a portion of the therapeutic payload into tissue.

10. The capsule device as in claim 7, wherein the delivery member is an injection needle, and wherein the therapeutic payload is provided as a liquid, gel or powder being expellable through the injection needle from a reservoir within the capsule housing.

11. The capsule device as in claim 8, wherein the actuator comprises an energy source associated with the delivery member, the energy source configured for powering the delivery member for being advanced from the capsule and into the lumen wall by movement of the actuator from the first configuration to the second configuration.

12. The capsule device as in claim 11, wherein the energy source of the actuator comprises a drive spring, such as a compression spring, the spring being strained or configured for being strained for powering the delivery member.

13. The capsule device as in claim 11, wherein the capsule device comprises a dissolvable firing member, the dissolvable firing member being at least partially dissolvable when subjected to a biological fluid, wherein the dissolvable firing member, when at least partially dissolved, permits release of energy from the energy source so that the delivery member is advanced from the capsule housing and into the lumen wall.

14. The capsule device as in claim 1, wherein the capsule device defines an ingestible capsule having a capsule housing shaped and sized to be ingested by a patient, such as a human patient.

15. The capsule device as in claim 5, wherein the capsule device is configured for release of therapeutic payload from the capsule into one of a lumen wall of the stomach, a lumen wall of the large intestines and a lumen wall of the small intestines of a patient.
